(19) United States
(12) Patent Application Publication
(10) Pub. No.: US 2002/0012698 A1
(43) Pub. Date: Jan. 31, 2002

(54) Magnetosomes, method for making and using

(76) Inventors: Edmund Bauerlein, Munchen (DE); Dirk Schuler, Satssfurt (DE); Regina Reszka, Schwanebeck (DE); Sabine Pauser, Berlin (DE)

Correspondence Address: GABRIEL P. KATONA L.L.P., 708 Third Avenue, 14th Floor, New York, NY, 10017, US

(21) Appl. No.: 09/764,970

(22) Filed: Jan. 18, 2001

Related U.S. Application Data
(62) Division of application No. 09/397,705, filed on Sep. 1, 1999, now Pat. No. 6,251,365.

(51) Int. Cl. A61K 48/00; A61K 9/127

(52) U.S. Cl. 424/450; 424/1.11; 514/44

(57) ABSTRACT

A magnetosome of a magnetite monocrystal having a diameter of at least 45 nm surrounded by a phospholipid membrane, and at least one therapeutic agent therein, a process of treating a tumoral disease. inflammatory process, or metabolic disease, and for removing diseased cells, by administering the aforesaid magnetosome.

### FIELD OF INVENTION

The invention relates to specific magnetosomes with magnetic particles of maximally 43 - 45 nm, and method for making and using them. The invention also relates to magnetoliposomes which can be obtained from these magnetosomes by liposomal encapsulation. The magnetoliposomes of the present invention are useful for medicinal applications.

### BACKGROUND

Superparamagnetic iron particles are known to be applied in medical diagnostics as NMR contrast agents or in the form of immunoconjugates or as synthetic drug carriers. Matsunaga et al. described in 1989 magnetosomes obtained from the magnetic bacterium Magnetospirillum spec. ABMI (JP7-241192-A) and their use. However, these magnetosomes have the disadvantage that they are comparatively large, thus bringing about the danger of embolisms.

### DESCRIPTION OF THE INVENTION

The object of the present invention is to provide specific magnetosomes which are smaller than those known, thus improving their medical use for reaching the envisaged targets in the body of the patient and also with reduced danger of embolisms.

It was detected that magnetosomes with magnetic particles <50 nm are contained in the bacterium Magnetospirillum gryphiswaldense. To our surprise, it was possible to produce these specific magnetosomes of the magnetic bacterium Magnetospirillum gryphiswaldense on a semi-industrial scale.

Accordingly, the object of the invention are the magnetosomes themselves, their method of preparation and their use in medicine and pharmacy.

The magnetosomes of the present invention contain a magnetic oxide magnetite Fe₃O₄ monocrystal with a maximum diameter of 43-45 nm surrounded by a phospholipid membrane. As a rule, they have a cubooctahedral shape.

The membrane is suitably phosphatidyl ethanolamine, phosphatidyl glycerol and phosphatidyl choline containing mainly the fatty acids palmitic acid, palmitoleic acid and oleic acid. The membrane suitably contains 53±6 % phosphatidyl ethanolamine, 38±6 % phosphatidyl glycerol and 8.9±5 % phosphatidyl choline where mainly the fatty acids palmitic acid (approx. 18.4 %), palmitoleic acid (approx. 25.6 %) and oleic acid (approx. 45.9 %) can be found.

A suitable embodiment of the present invention contains the magnetosomes as chains up to 100, most suitably 10-60 magnetosomes and with a cationic surface charge. This chain form of magnetosomes increases the probability that antibodies and therapeutic agents will be correctly bound to them and become effective.

In addition, these are also magnetosomes with additionally covalently bound antibodies or therapeutic agents bound to the magnetosome membrane through respective reactive groups.

The invention also comprises a method for preparing these new magnetosomes. They are isolated from the magnetic bacterium Magnetospirillum gryphiswaldense according to a new fermentation method. For this purpose a new simple culture medium of 0.3 g of KH₂PO₄, 1 g of Na acetate, 1 g of a soybean peptone (sold by Merck), 0.1 g of NH₄Cl, 0.1 g of yeast extract, at pH 6.9, which does not contain a complexing agent for iron is suitably used. The concentration of oxygen in the medium is maintained below 2 %, later Na acetate and FeSO₄ are added. After approx. 30 hours the magnetic cells can be gathered. After subjecting the cells to a lysis the magnetosomes are obtained in a high output according to a new method by separating them from cell fragments and cell sap in a magnetic separation column by a strong, powerful permanent magnet (Sm-Neodyn) and purifying them by washing.

Furthermore, magnetosomes according to the present invention are packed in liposomes, forming themselves liposomes with other lipids or bound to the surface of liposomes. Such liposomes are

(i) classical liposomes (MLV, SUV, LUV);

(ii) stealth liposomes (PEG);

(iii) micellar systems (e.g. SDS, triton, sodium cholate);

(iv) immunoliposomes containing e.g. antibodies or fab fragments against antigens associated with diseases or adhesion molecules bound to the surface of the liposomes;

(v) cationic liposomes (DAC-Chol, DOCSPER); and

(vi) fusogenic liposomes (reconstituted fusion proteins in liposomes).

Magnetoliposomes are prepared according to methods known per se, as e.g. described in German patents Nos. 41 34 158; 44 30 593; 44 46 937; and 196 31 189 with the magnetosomes being suitably added to the initial lipids.

The suitable modifications of magnetoliposomes and magnetosomes according to the present invention are represented in Table 1 below.

TABLE 1

| | | Magnetoliposomes | | |
|---|---|---|---|---|
| | | ↓ | | |
| | | | Cationic | Fusiogenic |
| Classical | Stealth | Immuno | e.g. | e.g. |
| MLV | PEG | anti-CEA | DAC Chol/DOPE | HN, F protein |
| SUV | | anti Thy1.1 | SP Chol/DOPE | (Sendai virus) |
| LUV | | anti CD44 | DAC-Quat. | → [pH 7] |
| (REV) | | anti CD54 | Chol/DOPE | synthetic |
| | | anti CD56 | DOCSPER | fusion |
| | | anti CD30 | | proteins |
| | | anti CD31 | | HA influenza |
| | | | | virus [pH 5,2] |
| | | | | Cochleates |

| | |
|---|---|
| | Magnetosomes |
| | ↓ |
| Immuno | Gene or antisense oligonucleotide |
| anti CEA | or ribozyme modified |
| anti CD44 | |
| anti | |
| CD54, | |
| CD56 | |
| anti CD30 | |

The magnetosomes and magnetoliposomes according to the invention can contain specific antibodies and one or a few therapeutic agents chemically coupled to their surfaces and enclosed, i.e. encapsulated radionuclides.

In addition, they, together with genetic material such e.g. plasmids, therapy genes, antisense oligonucleotides, ribozymes or gene diagnostic agents, can form cationic complexes suited for the transfer of genes.

These magnetosomes and magnetoliposomes (these terms being used interchangeably herein) according to the invention have a comprehensive spectrum of application. Owing to their magnetic properties they are used per se (also unmodified) as contrast agents for NMR examinations and as markers for mapping magnetic susceptibilities such as by a SQUID biomagnet meter, and also as diagnostic agents for the detection of various diseases, and foci of inflammatory or therapeutic agents as e.g. for purging (taking out diseased cells), as diagnostic agents for tumoral diseases or in lymphography, for inflammatory processes, for multiple sclerosis, Alzheimer disease and for Parkinson's disease, or as a therapeutic agent against tumoral diseases, inflammatory processes, and metabolic diseases.

Diagnostic agents are suitably used in the form of immunomagnetosomes or immunomagnetoliposomes. For this, antibodies or fab fragments against antigenes associated with diseases or adhesion molecules or ligands are covalently coupled to the magnetosome and magnetoliposome membrane through respective groups, suitably to phosphatidyl ethanolamine contained in the membrane through spacers of differing lengths.

In particular, they are used as diagnostic agents for the detection of tumoral diseases or in lymphography, with among others anti CEA, anti CD44 being coupled to the magnetosome membrane or magnetoliposome membrane as a reagent.

These antibody coupling products are also suited for detecting inflammatory processes such as arthroses (suitably with anti CD54, anti CD56) or for detecting multiple sclerosis or Alzheimer's disease (suitably anti-B-amyloid, anti APOE4), Hogkin lymphoma cells (suitably with anti CD30) and Parkinson's disease.

The magnetosomes according to the invention are particularly well suited for diagnostic applications.

It is necessary to use magnetoliposomes simultaneously to bring a therapeutic substance in relevant quantities to the target location. They are not only suited for coupling but also for enclosing therapeutic agents. In the case of magnetosomes therapeutic agents can be coupled only with a spacer being interconnected.

According to the present invention an essential possibility of use is that therapeutic agents are coupled (magnetosomes) or coupled or enclosed (magnetoliposomes). These therapeutic agents can be enclosed in the membrane or in the aqueous interior of the liposomes depending on lipophilicity or hydrophilicity.

Thus, the following suitable coupling variants are obtained according to the invention:

the therapeutic agent(s) is (are) coupled to the magnetosome or enclosed in the membrane;

the therapeutic agent(s) is (are) coupled to the magnetosome or enclosed in the membrane and packed in liposomes;

the magnetosome is packed as liposome and this or the therapeutic agents are enclosed in the aqueous interior of the liposomes; and

therapeutic agents are coupled to the magnetosome or enclosed in the membrane, the magnetosome is packed in liposomes and at least one further therapeutic agent is enclosed in the aqueous or lipophilic interior of the liposomes.

Important therapeutic agents that can be considered for this purpose include chemotherapeutic agents such as carboplatin or taxol, and radiotherapeutic agents such as yttrium, iodine, technetium or boron, and also therapy genes such as suicide genes, antisense oligonucleotides, ribozymes or cytokine genes can be coupled in this manner.

The invention enables a broad scope of medical application. The essential advantage of the magnetosomes and magnetoliposomes according to the invention enables that metastases can be better reached in the body and detected early, their enrichment in the lymphatic vessels is improved, and blood-brain barriers are better overcome by the new particles which is of particular importance to the detection of Alzheimer's plaques and the diagnosis of brain tumors.

The invention is explained in greater detail by the following examples of thereof.

### Example 1

Obtaining magnetosomes

To obtain magnetosomes in masses the cells of the magnetic bacterium Magnetospirillum gryphiswaldense were bred in a 100 [symbol] fermeter (LP 352, Bioeng. AG) at 30° C. in a culture medium of the following composition (per 1000 [symbol] ): 0.3 g of KH₂PO₄, 1 g of Na acetate, 1 g of soybean peptone (Merck), 0.1 g of NH₄Cl, 0.1 g of yeast extract, pH 6.9. Inoculation was effected by adding 5 [symbol] of pre-culture to 70 [symbol] of the medium. Aeration was regulated by stirring and input of compressed air so that the concentration of oxygen in the medium did not exceed 2 % of saturation. 70 g of Na acetate and iron sulfate were added to a concentration of 100 μM with the OD_{400=0.55.} After approx. 30 hours it was possible to gather magnetic cells.

The cells were centrifuged and washed. After the cell extract passed the French press three times and was subsequently subjected to a low-run centrifuging it was put into 20 mM HEPES/4 mM EDTA through a magnetic separation column (Miltenyi Biotec). The column was exposed to the magnetic field of a strong permanent magnet (Sm-Neodyn) to separate the magnetic particles. This produced a strong inhomogeneous magnetic field in a magnetizable column material for a specific binding of the magnetic particles. The magnetosomes were washed in the column with 20 mM HEPES/200 mM NaCl to remove specifically associated pollution. After having been washed with 20 mM HEPES the magnetosomes were flushed from the column after removing the magnetic field. To separate potentially available membrane contaminations, the magnetosome suspension was applied to a two-layer (50/55 % saccharose) sugar gradient and centrifuged in an ultracentrifuge with 25,000 rpm for 25 hurs. Potentially contained membrane components accumulated at the buffer-saccharose solution interphase whereas the magnetosome particles appeared as pellets on the bottom of the tube. The magnetosomes thus obtained appeared to be electronmicroscopically pure and showed a distinct lipid and protein pattern.

### Example 2

Use of magnetosomes

There were used magnetosomes from M. gryphiswaldense with an iron content of 1.35 g Fe/ [symbol] (determined by atomic absorption spectroscopy (AAS)). The relaxivities were determined by means of a Bruker Minispec pc 120 at 37° C. and 0.47 T as:

R₁=25.503 mM-1 * ₛ-1

R₂=226.179 mM-1 * ₛ-1.

The relaxivities, in particular R₂, are high as compared with various SPIOs (superparamagnetic iron oxide formulations). Comparable values were obtained only for SPIO-SUVs (small unilamellar vesicles).

The following in vivo experiment was carried out: The remaining substance quantity (0.4 m [symbol] ) was injected in vivo into the tail vein of a male WAG/RIJ (270 g rat) with a CC531 adenocarcinoma implanted into the liver. Thus, the animal received magnetosomes in a dose of 35.81 μmol Fe/kg of rat weight. The NMR examination was carried out with a Bruker Biospec BMT 24/40 instrument. Thereby, before, immediately after the infection and then at the time indicated in Table 2 nine 3 mm 5 layers and an enclosed external standard tube with the RARE sequence (TR =2500 ms, TE=20 ms, RF=8; NE=8) were taken up through the abdomen of the rat. The signal intensities in the liver and the tumour were measured in four different layers and were evaluated. The indicated weakening of the relative signal intensity SI ᵣₑₗ is calculated as follows:

SIᵣₑₗ=(SI_{post lip}./SI_{standard})(SI_{pre lip}/SI_{standard})

SI_{pre lip}=signal intensity before applying liposomes

SI_{post lip}=signal intensity after applying liposomes

SI_{standard}=signal intensity of the standard.

Given this comparatively low dose a signal reduction, up to already 90 % was reached in the liver, however in the tumor only weak SI reductions were observed (Table 2). This means that the tumor clearly stands out against the healthy liver tissue (FIG. 1).

TABLE 2

| | Tumor mean from | | | | | |
|---|---|---|---|---|---|---|
| | all > layers | | | Mean | | |
| pre | 1.00 | 1.00 | 1.00 | 1.00 | 1.00 | 0.00 |
| 5 min. | 0.93 | 0.89 | 0.91 | 0.98 | 0.93 | 0.04 |
| 15 min. | 1.00 | 0.96 | 0.98 | 1.02 | 0.99 | 0.03 |
| 31 min. | 1.04 | 0.99 | 0.99 | 1.06 | 1.02 | 0.04 |
| 48 min. | 1.01 | 0.98 | 0.98 | 1.06 | 1.00 | 0.04 |
| 65 min. | 1.00 | 0.98 | 0.98 | 1.13 | 1.02 | 0.07 |
| 82 min. | 0.95 | 0.94 | 0.93 | 1.05 | 0.97 | 0.06 |
| 113 min. | 0.93 | 0.86 | 0.91 | 1.10 | 0.95 | 0.10 |
| 24 h | 0.93 | 0.86 | 0.91 | 1.10 | 0.95 | 0.10 |
| 48 h | 1.05 | 1.00 | 1.02 | 1.14 | 1.05 | 0.10 |

| | | | | | | Standard |
|---|---|---|---|---|---|---|
| | Liver | | | | Mean | deviation |
| pre | 1.00 | 1.00 | 1.00 | 1.00 | 1.00 | 0.00 |
| 5 min. | 0.18 | 0.29 | 0.13 | 0.12 | 0.18 | 0.08 |
| 15 min. | 0.19 | 0.35 | 0.10 | 0.13 | 0.19 | 0.11 |
| 31 min. | 0.13 | 0.19 | 0.09 | 0.15 | 0.14 | 0.04 |
| 48 min. | 0.18 | 0.14 | 0.14 | 0.23 | 0.17 | 0.04 |
| 65 min. | 0.24 | 0.18 | 0.14 | 0.13 | 0.17 | 0.05 |
| 82 min. | 0.23 | 0.13 | 0.11 | 0.17 | 0.16 | 0.05 |
| 113 min. | 0.13 | 0.13 | 0.11 | 0.17 | 0.13 | 0.03 |
| 24 h | 0.11 | 0.13 | 0.11 | 0.17 | 0.13 | 0.03 |
| 48 h | 0.11 | 0.12 | 0.12 | 0.11 | 0.11 | 0.01 |

1. Specific magnetosomes consisting of a magnetic iron oxide magnetite Fe₃O₄ monocrystal with a diameter ≦45 nm and a phospholipid membrane surrounding this crystal.

2. Magnetosomes according to claim 1 wherein the membrane consists of phosphatidyl ethanolamine, phosphatidyl glycerol and phosphatidyl choline where mainly the fatty acids palmitic acid, paltitoleinic acid and oleic acid are contained.

3. Magnetosomes according to claims 1 and 2 wherein the membrane consists of 53±6 % phosphatidyl ethanolamine, 38±6 % phosphatidyl glycerol and 8.9±5 % phosphatidyl choline.

4. Magnetosomes according to claims 1 to 3 wherein they exist mainly as chains up to 100, suitably 10-60 magnetosomes and with a cationic surface charge.

5. Magnetosomes according to claims 1 to 4 wherein additionally antibodies or therapeutic agents, if necessary through respective reactive groups, are bound to the magnetosome membrane.

6. Magnetosomes according to claims 1 to 5 wherein they are contained packed in liposomes.

7. Magnetosomes according to claims 1 to 6 wherein they are contained packed in classical liposomes, stealth liposomes, micellar systems, immunoliposomes, cationic liposomes or fusogenic liposomes.

8. Magnetosomes according to claims 1-4 and 6-7 wherein they show additionally specific antibodies chemically coupled to their surface.

9. Magnetosomes according to claims 1-4 and 6-7 wherein they contain additionally one or a few therapeutic agents enclosed (encapsulated).

10. Magnetosomes according to claims 1-4 and 6-7 wherein the contain additionally radionuclides enclosed (encapsulated).

11. Magnetosomes according to claims 1-4 and 6-7 wherein they, together with genetic material (e.g. plasmids), therapy genes, antisense oligonucleotides, ribozymes or gene diagnostic agents, contain cationic complexes suited for the transfer of genes.

12. Method for the preparation of specific magnetosomes according to claims 1 to 4 wherein they are isolated from the magnetic bacterium magnetospirillum gryphiswaldense using a simple culture medium which does not contain complexing agents for iron, with the oxygen concentration in the medium being maintained below 2 %, later Na acetate and FeSO₄ being added, the magnetic cells being gathered by centrifugation and subsequently after lysis of cells the magnetosomes being obtained by separation of the cell fragments and cell sap by means of a permanent magnet in a magnetic separation column.

13. Use of specific magnetosomes according to claims 1-4 and 6-7 as NMR contrast agent.

14. Use of specific magnetosomes according to claims 1-5 for purging ( taking out diseased cells ).

15. Use of specific magnetosomes according to claims 1-4 and 6-7 as diagnostic agents for tumour diseases or in lymphography, for inflammatory processes, for multiple sclerosis, Alzheimer disease and Parkinson's disease.

16. Use of specific magnetosomes according to claims 1-10 as a therapeutic agent against tumoral diseases, inflammatory processes and metabolic diseases.
